(19) United States
(12) Patent Application Publication
(10) Pub. No.: US 2026/0290551 A1
(43) Pub. Date: Sep. 24, 2026

(54) SYSTEMS AND METHODS FOR AI-DRIVEN MEAL ANALYSIS AND NUTRITION OPTIMIZATION

(71) Applicant: Paul Murff, Salt Lake City, UT (US)

(72) Inventor: Paul Murff, Salt Lake City, UT (US)

(21) Appl. No.: 19/575,314

(22) Filed: Mar. 23, 2026

Related U.S. Application Data
(60) Provisional application No. 63/776,379, filed on Mar. 24, 2025.

(51) Int. Cl. G16H 20/60

(52) U.S. Cl. CPC G16H 20/60

(57) ABSTRACT

A system and method for AI-driven nutrition analysis and optimal meal scoring utilizes a multi-factor algorithm to evaluate meals based on ingredient quality, food processing level, nutrient density, macronutrient and micronutrient balance, glycemic impact, and meal timing. The system applies temporal metabolic weighting to adjust nutrient values based on circadian metabolic capacity and calculates dynamic nutrient synergies using interaction matrices that model non-linear relationships between nutrients. A knowledge graph database stores biological entities as nodes and physiological relationships as edges, enabling multi-hop queries for determining nutrient interaction effects. A deterministic logic layer comprising truth, law, and logic layers validates AI-generated recommendations against verified biological facts and physiological rules. The system generates personalized meal scores, actionable recommendations, and Protection Reports analyzing food brands and nutrition science literature, while enabling users to share results with designated individuals for accountability.

### CROSS-REFERENCE TO RELATED APPLICATIONS

The present application claims priority to U.S. Provisional Application No. 63/776,379 filed Mar. 24, 2025, titled “SYSTEMS AND METHODS FOR AI-DRIVEN MEAL ANALYSIS AND NUTRITION OPTIMIZATION,” which is hereby incorporated by reference in its entirety.

### TECHNICAL FIELD

The embodiments generally relate to systems and methods for AI-driven nutrition analysis and meal planning optimization.

### BACKGROUND

Managing nutritional intake and meal planning is essential for maintaining optimal health and wellness across diverse populations and lifestyles. Conventional nutrition tracking applications predominantly focus on calorie counting and macronutrient breakdowns, which may not offer a comprehensive view of a meal's nutritional value or its alignment with long-term health goals. Existing solutions typically require users to manually input detailed food information or rely on barcode scanning to track meals, often resulting in inaccurate data capture and user fatigue. These limitations can undermine the effectiveness of diet management strategies and reduce user engagement.

While some digital health platforms and mobile applications provide personalized nutrition recommendations, they frequently overlook important factors such as food processing levels, nutrient combinations that enhance bioavailability, and the timing of meal consumption in relation to circadian rhythms. Current applications focus on individual food items rather than assessing complete meals holistically, missing critical insights into how foods interact within a meal to support optimal nutrient absorption and health outcomes. Additionally, many solutions fail to adapt dynamically to users' evolving health metrics, activity levels, or dietary restrictions.

Furthermore, although a few platforms offer basic meal scoring or adherence tracking, they often lack integrated AI-driven analysis and actionable reporting to guide users in improving their nutritional habits. Users require a unified, intelligent system that simplifies meal planning, enhances accountability, and fosters long-term healthy eating behaviors without the complexity of traditional calorie-tracking applications.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

A system and method for AI-driven nutrition analysis and optimal meal scoring is disclosed. The system utilizes an intelligent algorithm to evaluate and score complete meals based on a variety of factors, including food processing levels, nutrient density, meal timing, and ingredient quality. The system leverages machine learning and artificial intelligence to provide personalized nutritional assessments and actionable recommendations that support long-term health and dietary adherence.

The AI-powered platform allows users to create a personalized profile by inputting biometric data, dietary preferences, and health goals. Users can build meals using an intuitive interface that includes meal cards representing curated meal options with corresponding nutritional highlights and scores. The system generates a personalized meal score for each meal, providing real-time feedback and insights into how the meal aligns with the user's health objectives. The platform further incorporates gamification to encourage user engagement, rewarding adherence and progress through an interactive meal-building experience.

The system applies temporal metabolic weighting to adjust nutrient values based on circadian metabolic capacity and consumption time of day, addressing the limitation of conventional systems that assign static nutritional values regardless of when food is consumed. The system also calculates dynamic nutrient synergies using interaction matrices that model non-linear relationships between nutrients within a meal, capturing beneficial combinations such as vitamin C enhancement of iron absorption and piperine enhancement of curcumin bioavailability that conventional calorie-tracking applications overlook.

The system integrates AI-driven Protection Reports that automatically analyze nutrition science literature, brand claims, and emerging dietary trends. These reports provide users with transparent and unbiased insights into food choices, including warnings about potentially misleading marketing practices or health risks associated with certain ingredients. The platform empowers users to make informed decisions by offering periodic or on-demand assessments of their dietary patterns and meal choices.

In addition, the system allows users to share their meal data and reports with designated individuals, such as family members, nutrition coaches, or healthcare providers. The system supports real-time updates and accountability scoring, tracking users' adherence to their personalized meal plans while simplifying the logging process to focus on the quality and health impact of foods consumed. In various embodiments, the platform includes capabilities for integrating third-party nutrition databases, fitness trackers, and wearable devices to enrich user data and enhance personalization.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a system architecture diagram depicting the overall network topology and component interconnections for the AI-driven nutrition analysis and optimal meal scoring system, according to some embodiments;

FIG. 2 illustrates a detailed block diagram of the application server architecture showing the internal module organization and data flow pathways for the nutrition analysis platform, according to some embodiments;

FIG. 3 illustrates a schematic diagram of the multi-factor algorithm architecture depicting the variable categories and evaluation modules used to generate personalized meal scores, according to some embodiments;

FIG. 4 illustrates a knowledge graph database schema diagram showing the node types, relationship edges, and data structures used for storing and querying nutritional interaction data, according to some embodiments;

FIG. 5 illustrates a block diagram of the deterministic logic layer architecture showing the triple-layer validation system for constraining AI-generated nutritional recommendations, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

A system for AI-driven nutrition analysis and optimal meal scoring may comprise at least one user computing device in operable connection with a user network. The user computing device may be a smartphone, tablet computer, personal computer, wearable device, or any other computing device capable of executing application software and communicating over a network. The user network may include the Internet, a local area network, a wide area network, a cellular network, or any combination thereof. The user computing device may communicate with an application server through standard network protocols including but not limited to HTTP, HTTPS, WebSocket, or proprietary protocols designed for real-time data exchange.

The application server may be in operable communication with the user network and may be configured to host an application program for generating personalized meal scores and providing nutritional guidance. The application server may comprise one or more physical servers, virtual machines, cloud computing instances, or serverless computing functions distributed across multiple geographic regions. The application server may utilize load balancing, auto-scaling, and fault tolerance mechanisms to ensure high availability and responsiveness. The application program may be implemented using a microservices architecture where discrete functional components communicate through application programming interfaces. The application program may be in operable communication with one or more processors coupled to a memory, wherein the memory may include volatile memory such as random access memory and non-volatile memory such as solid-state drives or hard disk drives for persistent data storage.

The one or more processors may be configured to enable a user to create a personalized account and input personal data. The personalized account may be created through a registration process that collects authentication credentials such as email addresses, usernames, and passwords, or through integration with third-party authentication providers using protocols such as OAuth or OpenID Connect. Personal data may include biometric data such as height, weight, body composition measurements, resting heart rate, blood pressure, and other physiological measurements. Health goals may include weight management objectives, athletic performance targets, disease prevention strategies, energy optimization, cognitive enhancement, and longevity-focused outcomes. Dietary preferences may include food allergies, intolerances, religious dietary restrictions, ethical dietary choices such as vegetarianism or veganism, cuisine preferences, and ingredient preferences or aversions. Activity levels may be characterized as sedentary, lightly active, moderately active, very active, or extremely active, and may be quantified through integration with fitness trackers and wearable devices that measure steps, active minutes, exercise sessions, and energy expenditure.

The one or more processors may be configured to receive and process meal data input by the user. The meal data may comprise ingredients, preparation methods, and meal timing. Ingredients may be identified through text entry with autocomplete suggestions drawn from a food composition database containing nutritional profiles for hundreds of thousands of food items. Alternatively, ingredients may be identified through barcode scanning using the camera of the user computing device, image recognition using machine learning models trained to identify foods from photographs, or voice input processed through natural language understanding. The food composition database may include data sourced from government databases such as the United States Department of Agriculture FoodData Central, open-source databases such as Open Food Facts, and proprietary databases compiled from food manufacturers and restaurants. Each food item in the database may be represented as a multi-dimensional object containing identity data including taxonomic classification and origin, chemistry data including nutrient density and glycemic index, interaction keys defining required synergies and triggered antagonisms, and processing state data defining different bioavailability characteristics for raw versus cooked preparation states.

Preparation methods may include cooking techniques such as boiling, steaming, roasting, grilling, frying, sauteing, baking, and raw preparation. Each preparation method may modify the bioavailability and nutritional properties of ingredients. For example, cooking may increase the bioavailability of certain nutrients such as lycopene in tomatoes while decreasing the availability of heat-sensitive nutrients such as vitamin C. The system may maintain preparation state data that defines different bioavailability characteristics for each food based on its processing state. Meal timing may include the date and time of consumption, which may be entered manually by the user or determined automatically based on when the meal is logged in the application.

The one or more processors may be configured to generate a meal score based on a multi-factor algorithm. The multi-factor algorithm may evaluate at least ingredient quality, food processing level, nutrient density, macronutrient and micronutrient balance, glycemic index, and meal timing. Ingredient quality may be assessed based on sourcing information such as organic certification, grass-fed designation for animal products, wild-caught designation for seafood, and geographic origin. Food processing level may be categorized according to a classification system that distinguishes between unprocessed or minimally processed foods, processed culinary ingredients, processed foods, and ultra-processed foods. Ultra-processed foods may receive scoring penalties based on the presence of ingredients typically found only in industrial food manufacturing such as hydrogenated oils, high-fructose corn syrup, artificial colors, artificial flavors, and emulsifiers.

Nutrient density may be calculated as the ratio of beneficial nutrients to caloric content, accounting for vitamins, minerals, fiber, essential fatty acids, and phytonutrients per calorie. Macronutrient balance may be evaluated against user-specific targets for protein, carbohydrates, and fats, with the targets adjusted based on user health goals, activity levels, and metabolic state. Micronutrient sufficiency may be assessed by comparing intake against reference daily intakes, estimated average requirements, recommended dietary allowances, adequate intakes, and tolerable upper intake levels, with the specific reference values adjusted based on user age, sex, and physiological status. Glycemic impact may be evaluated based on the glycemic index and glycemic load of carbohydrate-containing foods in the meal, with adjustments for the presence of fiber, protein, and fat that may moderate glycemic response.

The multi-factor algorithm may utilize a comprehensive variable system comprising at least one hundred twenty variables across categories including macronutrients, micronutrients, bioactive compounds, food quality, timing context, meal composition, personalization factors, and functional nutrition parameters. Macronutrient variables may include total protein, complete protein score, essential amino acid profile, branched-chain amino acid content, leucine threshold for muscle protein synthesis, total carbohydrates, fiber content, sugar content, added sugar content, glycemic index, glycemic load, total fat, saturated fat, monounsaturated fat, polyunsaturated fat, omega-3 fatty acid content, omega-6 fatty acid content, and omega-6 to omega-3 ratio. Micronutrient variables may include vitamins A, C, D, E, K, B1, B2, B3, B5, B6, B7, B9, and B12, as well as minerals including calcium, magnesium, potassium, sodium, iron, zinc, copper, manganese, selenium, phosphorus, iodine, and chromium. Bioactive compound variables may include polyphenols, flavonoids, carotenoids, glucosinolates, organosulfur compounds, and prebiotic fibers.

The one or more processors may be configured to apply temporal metabolic weighting to adjust nutrient values based on circadian metabolic capacity and consumption time of day. Temporal metabolic weighting may be implemented using circadian multipliers for different nutrient classes based on time-dependent metabolic capacity. The temporal metabolic function may be expressed as a base value multiplied by a sum of sinusoidal functions, where each sinusoidal function represents the circadian rhythm for a particular nutrient class with an amplitude representing the magnitude of circadian variation and a phase shift representing the optimal absorption time. For protein, the temporal weighting may increase protein value during morning hours corresponding to muscle synthesis peaks, with an increase of approximately thirty percent between seven o'clock in the morning and ten o'clock in the morning. For carbohydrates, the temporal weighting may decrease carbohydrate value during evening hours corresponding to reduced insulin sensitivity, with a decrease of approximately forty percent after seven o'clock in the evening. For calcium, the temporal weighting may increase absorption value during evening hours corresponding to parathyroid hormone rhythm, with an increase of approximately fifty percent during evening hours.

The temporal metabolic weighting may account for the user's chronotype, which may be determined through questionnaires assessing sleep-wake preferences or through analysis of activity patterns from wearable devices. Users with an early chronotype may have circadian peaks shifted earlier in the day, while users with a late chronotype may have circadian peaks shifted later. The temporal metabolic weighting may also account for shift work schedules, jet lag, and other disruptions to normal circadian patterns.

The one or more processors may be configured to calculate dynamic nutrient synergies using interaction calculations that model non-linear relationships between nutrients within the meal. The synergy matrix may be expressed as a product of synergy factors, where each synergy factor represents the interaction between a pair of nutrients. The interaction calculation for a nutrient pair may follow a saturation kinetics model where the synergy benefit increases with the product of the two nutrient concentrations but approaches a maximum value asymptotically. For example, the interaction calculation for vitamin C enhancement of iron absorption may be expressed as one plus a coefficient multiplied by the product of vitamin C concentration and iron concentration divided by a saturation constant plus the product of the concentrations. The coefficient may be approximately three point two, reflecting the substantial enhancement of non-heme iron absorption in the presence of vitamin C.

The interaction calculations for specific nutrient pairs may include vitamin C enhancement of iron absorption, calcium and vitamin D interaction for bone health, fat-soluble vitamin absorption enhancement in the presence of dietary fats, piperine enhancement of curcumin bioavailability, and zinc and copper competition for absorption. The piperine enhancement of curcumin bioavailability may be expressed as one plus twenty multiplied by the product of curcumin concentration and piperine concentration divided by one plus curcumin concentration, reflecting the approximately twenty-fold increase in curcumin bioavailability when consumed with black pepper containing piperine. The synergy matrix may be stored in a knowledge graph database where nutrients are represented as nodes and synergistic or antagonistic relationships are represented as edges with associated interaction coefficients.

The one or more processors may be configured to provide real-time feedback and actionable recommendations to the user for improving the nutritional quality of the meal based on the generated meal score. The real-time feedback may be presented through an interactive user interface that displays the meal score as a numerical value on a scale from zero to one hundred, with accompanying visual indicators such as color coding or graphical meters. The meal score may be decomposed into component sub-scores representing different aspects of nutritional quality such as macronutrient balance, micronutrient sufficiency, food quality, inflammatory potential, satiety prediction, and energy sustainability. Each sub-score may be displayed with explanatory text describing the factors contributing to the score and specific suggestions for improvement.

The actionable recommendations may include suggestions for adding specific foods to address nutrient deficiencies, substituting higher-quality alternatives for lower-quality ingredients, adjusting portion sizes to improve macronutrient balance, and modifying eating sequence to optimize glycemic response. The recommendations may be generated using a recommendation engine that considers the user's dietary preferences, available ingredients, cooking skills, time constraints, and budget. The recommendation engine may utilize collaborative filtering to suggest foods that similar users have found effective for improving meal scores. The recommendations may be presented as specific food swaps, where a lower-scoring ingredient may be replaced with a higher-scoring alternative that serves a similar culinary function.

The one or more processors may be configured to generate and display Protection Reports that analyze food brands, nutrition science literature, and market trends to provide users with insights regarding the health impact and marketing claims of selected food items. The Protection Reports may be generated periodically or on demand based on user requests or automated triggers such as the user scanning a new product or adding a new brand to their meal log. The Protection Reports may include analysis of ingredient sourcing, food additives, and potential health risks associated with consumption of specific food items or brands.

The Protection Reports may utilize natural language processing to analyze product packaging claims and marketing materials, comparing stated benefits against scientific literature stored in a vector database. The vector database may store embeddings of scientific papers, clinical trials, and regulatory documents that can be semantically searched to find evidence supporting or contradicting product claims. The Protection Reports may generate a validity index that quantifies the degree of alignment between marketing claims and scientific evidence, with specific identification of potentially misleading claims. The Protection Reports may also identify emerging dietary trends and evaluate the scientific basis for popular nutritional claims, helping users distinguish evidence-based recommendations from fads or marketing-driven trends.

The one or more processors may be configured to enable users to share meal scores, meal cards, and Protection Reports with designated individuals or communities to facilitate accountability and engagement in nutritional goals. The sharing functionality may support transmission to accountability partners, family members, healthcare providers, or nutrition coaches through secure messaging, email, or integration with third-party communication platforms. The shared content may include the personalized meal score, a visual representation of the meal composition, specific nutritional highlights, and suggestions for improvement. Users may configure privacy settings to control what information is shared and with whom, including options to share only aggregate scores over time periods rather than individual meal details.

The application program may include an interactive user interface that presents meals as visual meal cards. Each meal card may display nutritional highlights including the overall meal score, sub-scores for key nutritional dimensions, macronutrient distribution, and notable micronutrients. The meal cards may also display suggested improvements for increasing the meal score, presented as specific actionable steps the user can take. The interactive user interface may implement a guided workflow that walks users through meal composition in a step-by-step manner, prompting for protein sources, vegetables, carbohydrates, and healthy fats in a sequence designed to encourage balanced meal construction.

The application program may integrate with third-party fitness trackers and wearable devices to dynamically adjust meal score calculations based on real-time activity levels and biometric data. The integration may utilize application programming interfaces provided by fitness tracker manufacturers or health data platforms such as Apple HealthKit, Google Fit, or similar services. The real-time activity data may include steps taken, active minutes, heart rate, heart rate variability, sleep duration, sleep quality, and energy expenditure calculated from movement sensors and heart rate monitors. The integration may enable dynamic adjustments such as increasing protein and carbohydrate targets following intense exercise sessions, adjusting caloric targets based on measured energy expenditure, and modifying nutrient timing recommendations based on workout schedules.

The one or more processors may be further configured to apply cascading bioavailability functions that calculate nutrient availability based on consumption sequence of foods within the meal. The cascading bioavailability function may be expressed as a product of sequence factors, where each sequence factor depends on the time delay between consumption of different food components. The sequence effects may account for the physiological benefits of consuming foods in a specific order, such as the fiber-first protocol where consuming fiber-rich foods before protein and carbohydrates creates a viscous matrix in the stomach that slows gastric emptying and reduces glucose absorption rate.

Research has demonstrated that consuming fiber before protein and carbohydrates may result in approximately twenty-nine percent reduction in postprandial glucose excursion, improved satiety hormone response including increased glucagon-like peptide one secretion of approximately fifteen percent, and reduced glycemic impact with approximately thirty-seven percent lower insulin response. The cascading bioavailability functions may model these time-dependent absorption effects using exponential functions that approach maximum effect as the time delay increases. The system may prompt users to log the order in which they consume different components of a meal, or may infer order based on cultural eating patterns and user-specified preferences.

The one or more processors may be further configured to adapt the meal score based on an adaptive personal metabolic state that incorporates current metabolic state variables including glucose levels, recent exercise activity, sleep quality, and stress indicators. The adaptive personal metabolic state may be expressed as a baseline value multiplied by genetic factors and summed with current state modifiers. The current state modifiers may include adjustments for post-exercise metabolic state, where protein value may be increased by approximately forty percent and carbohydrate value may be increased by approximately sixty percent during the post-exercise anabolic window when muscle glycogen stores are depleted and muscle protein synthesis rates are elevated.

The current state modifiers may also include adjustments for ketogenic metabolic state, where fat value may be increased by approximately thirty percent and carbohydrate intake may receive a penalty of approximately fifty percent to maintain ketosis. Sleep deprivation may trigger increased antioxidant value of approximately eighty percent to compensate for increased oxidative stress associated with sleep debt. Stress indicators derived from heart rate variability or user-reported stress levels may trigger adjustments to nutrient targets, such as increased magnesium and B-vitamin requirements during periods of elevated stress. The metabolic state data may be received from integrated wearable devices that measure continuous glucose levels, activity patterns, sleep metrics, and physiological indicators of stress.

The system may further comprise a knowledge graph database structured to store biological entities as nodes and physiological relationships as edges. The knowledge graph database may store approximately six hundred twenty thousand nodes representing foods, nutrients, body functions organized into functional systems, hormones, and microbiome components including bacterial species and their metabolites. The knowledge graph database may store approximately four to five million relationships connecting these nodes, representing relationships such as food-contains-nutrient, nutrient-synergizes-with-nutrient, food-supports-body-system, and nutrient-deficiency-cascades-to-symptom. The knowledge graph database may enable multi-hop queries for determining nutrient interaction effects and synergistic relationships between food components, allowing the system to trace chains of causation such as fiber intake leading to short-chain fatty acid production by gut bacteria leading to reduced neuroinflammation leading to improved cognitive function.

A computer-implemented method for AI-driven nutrition analysis and optimal meal scoring may comprise receiving, by one or more processors, user profile data comprising biometric data, health conditions, dietary preferences, fitness goals, and activity levels. The method may comprise receiving, by the one or more processors, meal data input by a user, the meal data comprising a plurality of ingredients, preparation methods, and a meal consumption time. The method may comprise applying, by the one or more processors, temporal metabolic weighting functions to adjust nutritional values of the plurality of ingredients based on circadian rhythm optimization and the meal consumption time.

The method may comprise calculating, by the one or more processors, dynamic nutrient synergies between the plurality of ingredients using a synergy matrix that models non-linear nutrient interaction effects. The method may comprise generating, by the one or more processors, a personalized meal score based on evaluation of at least ingredient quality, food processing level, nutrient density, macronutrient balance, micronutrient sufficiency, glycemic impact, and the calculated dynamic nutrient synergies. The method may comprise applying, by the one or more processors, detractor penalties to the personalized meal score based on presence of identified harmful ingredients or nutrient antagonisms within the meal.

Detractor penalties may be applied multiplicatively to the meal score, such that the presence of multiple detractors compounds their negative effect. Detractor penalties may be applied for ultra-processed food ingredients, excessive added sugars, excessive sodium, artificial trans fats, ingredients known to trigger inflammatory responses, and ingredients to which the user has indicated sensitivity or intolerance. The detractor penalty system may implement a hyper-palatability detection algorithm that identifies combinations of high fat and high sugar content that may bypass normal satiety signaling, applying additional penalties to meals that exhibit these characteristics.

The method may comprise providing, by the one or more processors, real-time feedback to the user comprising the personalized meal score and actionable recommendations for improving nutritional quality of the meal. The method may comprise storing the meal data and associated personalized meal score in a database for tracking user dietary patterns over time. The stored data may enable longitudinal analysis of dietary patterns, identification of trends and patterns in meal scoring, and generation of personalized insights based on historical performance. The database may utilize a single-table design pattern optimized for fast transactional queries, with entities including user profiles, meal records, challenge records for behavioral interventions, and meal plan records for future meal planning.

The operations may further comprise mapping standard anatomical systems to functional engineering vectors comprising assimilation, defense, transport, biotransformation, energy, communication, and structure functional categories. The assimilation system may encompass the digestive system and govern the intake of fuel and the integrity of the intestinal barrier. The defense system may encompass the immune system and integumentary system, functioning as a biological firewall that rejects foreign entities. The transport system may encompass the cardiovascular system and function as a logistics network that moves cargo including oxygen, nutrients, and hormones throughout the body.

The biotransformation system may encompass the urinary system and liver function, operating as a waste management system that filters blood and metabolizes toxins for excretion. The energy system may encompass the respiratory system and cellular metabolism including mitochondrial function, operating as a power plant that converts nutrients and oxygen into adenosine triphosphate. The communication system may encompass the nervous system, endocrine system, and reproductive system, functioning as a signal network that transmits both fast neural signals and slow hormonal signals. The structure system may encompass the skeletal and muscular systems, providing mechanical integrity through bone leverage and muscle torque.

The operations may further comprise calculating synergistic pathway effects between functional systems to determine systemic health impacts of the meal composition. The synergistic pathways may include the gut-brain axis connecting the assimilation system to the communication system, where dietary fiber fermentation by gut bacteria produces short-chain fatty acids such as butyrate that cross the blood-brain barrier and reduce neuroinflammation. The synergistic pathways may include the oxygen-mitochondria axis connecting the transport system to the energy system, where dietary nitrates from foods such as beetroot convert to nitric oxide, causing vasodilation that improves oxygen delivery and thereby increases adenosine triphosphate production.

The synergistic pathways may include the detox-defense axis connecting the biotransformation system to the defense system, where activation of liver detoxification pathways by sulforaphane from cruciferous vegetables reduces the toxic burden on the immune system and thereby reduces systemic inflammation. The system may implement a law of limiting factors that constrains the overall health score based on the weakest functional system, such that a failing transport system caps the energy score regardless of nutrient intake because nutrients cannot be delivered to cells without adequate circulation.

The operations may comprise applying a deterministic logic layer to constrain nutritional analysis outputs. The deterministic logic layer may comprise a hierarchy of physiological rules including universal laws governing chronobiology and thermodynamics, conditional laws dependent on user metabolic state, and environmental laws governing toxicity thresholds. The universal laws may include the law of energy balance stating that mass change equals energy input minus energy output, the law of essentiality stating that the sum of essential nutrients must meet or exceed adjusted reference daily intakes, and the law of homeostasis stating that inputs trigger regulatory responses to maintain physiological equilibrium.

The conditional laws may include an anabolic threshold law stating that protein intake at or above approximately thirty grams per meal triggers muscle protein synthesis, an insulin sensitivity law stating that sleep debt or high stress reduces carbohydrate tolerance, and an absorption efficiency law stating that nutrient absorption is enhanced by specific synergistic pairings. The environmental laws may include toxicity threshold rules that block recommendations that would exceed tolerable upper intake levels for any nutrient or that would expose the user to harmful levels of contaminants such as heavy metals or pesticide residues.

The deterministic logic layer may comprise a triple-layer architecture including a truth layer containing verified biological facts as read-only data, a law layer containing deterministic rules encoded as logic gates for validating outputs, and a logic layer comprising an inference engine that synthesizes the truth layer and law layer against user-specific metabolic context. The truth layer may function as a ground truth database containing verified biological facts derived from clinical literature, where facts are stored as immutable data that cannot be modified by the generative AI components of the system. This architecture may prevent hallucination by ensuring that all AI-generated recommendations are validated against verified facts before being presented to users.

The law layer may contain deterministic rules encoded as conditional logic gates that override any generative output. For example, if the generative AI component suggests raw spinach to a user with a history of kidney stones, the law layer may block this output and force recalculation because raw spinach contains high oxalate content that may exacerbate kidney stone formation. The logic layer may function as a processor that combines facts from the truth layer and rules from the law layer with the user persona to generate specific recommendations. The logic layer may implement a hallucination filter where the AI attempts to generate advice, the logic layer cross-references the advice against the law layer, and invalid outputs are blocked with a recalculating prompt to the generative component.

The truth layer may be designed to be versioned like software, allowing the system to incorporate new scientific consensus without disrupting the core logic. When scientific understanding evolves, such as revised understanding of the health effects of dietary cholesterol, the truth layer may be updated to a new version that reflects current consensus, and the logic layer may automatically recalculate scores based on the updated truth data. The system may also implement a heuristic strengthening mechanism that recursively updates the confidence weights of specific node interactions based on aggregated, anonymized user outcome data. If a pattern emerges where users who consume a particular food combination report specific outcomes, the system may increase the weight of the edge representing that relationship in the knowledge graph.

A non-transitory computer-readable storage medium may store computer-executable instructions that, when executed by one or more processors, cause the one or more processors to perform operations for AI-driven nutrition analysis and optimal meal scoring. The non-transitory computer-readable storage medium may include magnetic storage media such as hard disk drives, optical storage media such as compact discs or digital versatile discs, solid-state storage media such as flash memory or solid-state drives, or cloud storage services that provide persistent storage accessible over a network. The computer-executable instructions may be organized as executable code modules, libraries, scripts, or containerized applications that implement the various functions described herein.

The knowledge graph database may comprise food nodes structured as multi-dimensional objects containing identity data, chemistry data including nutrient density and glycemic index, interaction keys defining required synergies and triggered antagonisms, and processing state data defining different bioavailability characteristics for raw versus cooked preparation states. The chemistry data may include nutrient profiles derived from compositional analysis, bioactive compound concentrations, and physical properties affecting digestion and absorption. The interaction keys may define positive synergies where the presence of another food or nutrient enhances bioavailability or biological effect, as well as antagonisms where another food or nutrient interferes with absorption or biological effect.

The systems and methods described herein provide a comprehensive framework for AI-driven nutrition analysis that accounts for temporal metabolic patterns, nutrient synergies and antagonisms, food processing effects, individual metabolic variation, and meal sequencing effects. The integration of a knowledge graph database with a deterministic logic layer enables the generation of personalized meal scores and actionable recommendations while preventing erroneous outputs through validation against verified biological facts and physiological rules. The functional system mapping enables holistic assessment of how meals affect interconnected physiological systems, moving beyond nutrient-level analysis to system-level health impact prediction.

Various implementations of the invention involve the technical field of AI-driven nutrition analysis and meal planning optimization including receiving, by one or more processors, user profile data comprising biometric data, health conditions, dietary preferences, fitness goals, and activity levels; receiving, by the one or more processors, meal data input by a user, the meal data comprising a plurality of ingredients, preparation methods, and a meal consumption time; applying, by the one or more processors, temporal metabolic weighting functions to adjust nutritional values of the plurality of ingredients based on circadian rhythm optimization and the meal consumption time; calculating, by the one or more processors, dynamic nutrient synergies between the plurality of ingredients using a synergy matrix that models non-linear nutrient interaction effects; generating, by the one or more processors, a personalized meal score based on evaluation of at least ingredient quality, food processing level, nutrient density, macronutrient balance, micronutrient sufficiency, glycemic impact, and the calculated dynamic nutrient synergies; applying, by the one or more processors, detractor penalties to the personalized meal score based on presence of identified harmful ingredients or nutrient antagonisms within the meal; providing, by the one or more processors, real-time feedback to the user comprising the personalized meal score and actionable recommendations for improving nutritional quality of the meal; and storing the meal data and associated personalized meal score in a database for tracking user dietary patterns over time, and are therefore necessarily rooted in computer technology. For example, the aforementioned steps are inherently computer-based and cannot be performed in the human mind.

The present invention amounts to more than merely implementing the generic computer as a tool to gather, analyze, and output data because the steps of the present method, system, or product improve the field of AI-driven nutrition analysis and meal planning optimization by applying a sinusoidal circadian multipliers to nutrient values based on consumption time, a synergy matrix using saturation kinetics calculations to model non-linear nutrient interactions, cascading bioavailability functions that calculate absorption based on eating sequence using exponential time-delay functions, and a knowledge graph database with approximately 620,000 nodes and 5 million relationship edges enabling multi-hop queries to trace nutrient interaction pathways through interconnected biological systems.

The system is not merely organizing human activity on generic hardware because it performs specific data transformations that have no manual analog. A human nutritionist cannot mentally compute the circadian phase-shifted absorption coefficients for dozens of nutrients simultaneously, calculate tensor-based synergy matrices across thousands of potential nutrient pairings in real-time, or traverse a multi-million-edge knowledge graph to identify cascading effects through seven functional biological systems. The deterministic logic layer implements a triple-layer architecture where a truth layer stores immutable verified biological facts, a law layer encodes physiological rules as logic gates that preemptively block invalid outputs, and a logic layer synthesizes these against user-specific metabolic context, this constitutes a specific technical solution for constraining AI-generated outputs to prevent hallucination, not a business method. The transformation of 120+ physiological variables through temporal weighting, synergy calculation, sequence optimization, and metabolic state adaptation into a single predictive bio-solvency metric represents a specific technical improvement in how computers process and synthesize nutritional data, producing an output that could not be achieved through mental steps or conventional nutritional calculations.

Additionally, the steps of the present invention would be impossible to accomplish on pen and paper due to the volume of data being communicated and received over a network in real-time. In particular, the speed at which the steps of the present invention occur to effectuate the disclosed method, system, or product would involve large-scale, continuous wireless communication of such data. That is, the steps of the present method, system, or product are impossible to accomplish on pen and paper, cannot be accomplished as a method of organizing human activity, and amount to significantly more than merely gathering, analyzing, and outputting data.

Implementations of the present invention include implementing (executing, running, or deploying) one or more artificial intelligence models on a computing device wherein the computing device executes the artificial intelligence model's algorithms and mathematical functions on computer hardware using machine learning libraries. The computing device implements the artificial intelligence model when it performs tasks like training, making predictions, applying the model to data, decision-making, classification, or generating outputs based on inputs. In particular, the speed at which an artificial intelligence model analyzes and transforms data to effectuate the disclosed method, system, or product would involve large-scale, continuous transformation of such data. As such, the present invention would be impossible to accomplish on pen and paper or in the human mind due to the volume of data being analyzed and transformed by the artificial intelligence model.

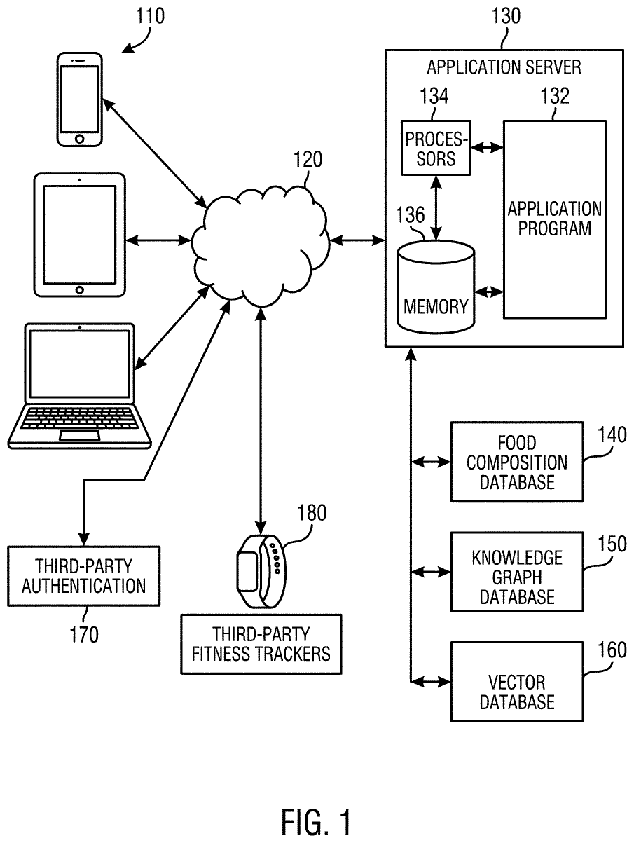

FIG. 1 illustrates a system architecture diagram 100 depicting the overall network topology and component interconnections for the AI-driven nutrition analysis and optimal meal scoring system, according to some embodiments. The system architecture enables seamless communication between user computing devices, application servers, and external services to provide comprehensive nutritional analysis and personalized meal scoring functionality.

User computing devices 110 are shown at the upper left portion of the diagram and may include smartphones, tablet computers, and personal computers capable of executing application software and communicating over a network. The user computing devices 110 represent the various client devices through which users interact with the AI-driven nutrition analysis platform. Each user computing device 110 may execute a client application that provides an interactive user interface for inputting meal data, viewing personalized meal scores, and receiving actionable nutritional recommendations. The user computing devices 110 are configured to capture meal data through multiple input modalities including text entry with autocomplete suggestions, barcode scanning using integrated cameras, image recognition for identifying foods from photographs, and voice input processed through natural language understanding.

A user network 120, depicted as a cloud symbol at the center-left portion of the diagram, provides the communication pathway connecting the user computing devices 110 to the application server 130. The user network 120 may include the Internet, local area networks, wide area networks, cellular networks, or any combination thereof. The user network 120 facilitates the transmission of meal data from user computing devices 110 to the application server 130 and the delivery of personalized meal scores and recommendations from the application server 130 back to the user computing devices 110. Standard network protocols including HTTP, HTTPS, WebSocket, or proprietary protocols designed for real-time data exchange may be utilized for communication across the user network 120.

The application server 130 is depicted as a rectangular block at the right side of the diagram and is configured to host an application program 132 for generating personalized meal scores and providing nutritional guidance. The application server 130 may comprise one or more physical servers, virtual machines, cloud computing instances, or serverless computing functions distributed across multiple geographic regions. The application server 130 utilizes load balancing, auto-scaling, and fault tolerance mechanisms to ensure high availability and responsiveness.

Within the application server 130, processors 134 are depicted as a component coupled to memory 136 and the application program 132. The processors 134 execute the computer-executable instructions of the application program 132 to perform the various nutritional analysis and meal scoring operations. The processors 134 are configured to enable users to create personalized accounts, receive and process meal data, generate meal scores based on the multi-factor algorithm, apply temporal metabolic weighting, calculate dynamic nutrient synergies, provide real-time feedback, generate Protection Reports, and enable sharing of meal data with designated individuals or communities.

The memory 136, shown within the application server 130, includes volatile memory such as random access memory for temporary data storage during processing operations and non-volatile memory such as solid-state drives or hard disk drives for persistent data storage. The memory 136 stores the application program 132, user profile data, meal records, and intermediate computational results generated during meal score calculations.

The application program 132, depicted within the application server 130, implements the core functionality of the AI-driven nutrition analysis system using a microservices architecture where discrete functional components communicate through application programming interfaces. The application program 132 coordinates the various processing modules, database interactions, and user interface components to deliver personalized nutritional assessments and actionable recommendations.

A food composition database 140 is shown at the lower right portion of the diagram and stores nutritional profiles for hundreds of thousands of food items. The food composition database 140 may include data sourced from government databases such as the United States Department of Agriculture FoodData Central, open-source databases such as Open Food Facts, and proprietary databases compiled from food manufacturers and restaurants. Each food item in the food composition database 140 is represented as a multi-dimensional object containing identity data, chemistry data including nutrient density and glycemic index, interaction keys defining required synergies and triggered antagonisms, and processing state data defining different bioavailability characteristics for raw versus cooked preparation states.

A knowledge graph database 150 is depicted below the food composition database 140 and is structured to store biological entities as nodes and physiological relationships as edges. The knowledge graph database 150 stores approximately six hundred twenty thousand nodes representing foods, nutrients, body functions organized into functional systems, hormones, and microbiome components including bacterial species and their metabolites. The knowledge graph database 150 stores approximately four to five million relationships connecting these nodes, representing relationships such as food-contains-nutrient, nutrient-synergizes-with-nutrient, food-supports-body-system, and nutrient-deficiency-cascades-to-symptom. The knowledge graph database 150 enables multi-hop queries for determining nutrient interaction effects and synergistic relationships between food components, allowing the system to trace chains of causation through interconnected biological systems.

A vector database 160 is shown at the bottom right of the diagram and stores embeddings of scientific papers, clinical trials, and regulatory documents that can be semantically searched to find evidence supporting or contradicting product claims. The vector database 160 supports the generation of Protection Reports by enabling natural language processing to analyze product packaging claims and marketing materials against scientific literature. The vector database 160 facilitates the calculation of validity indices that quantify the degree of alignment between marketing claims and scientific evidence.

Third-party authentication services 170 are depicted at the lower left portion of the diagram and provide authentication capabilities for user account creation and login. The third-party authentication services 170 support protocols such as OAuth or OpenID Connect for integration with external authentication providers. The third-party authentication services 170 enable users to create personalized accounts through a registration process that collects authentication credentials such as email addresses, usernames, and passwords, or through single sign-on using existing social media or enterprise identity providers.

Third-party fitness trackers 180 are shown adjacent to the third-party authentication services 170 and represent external wearable devices and fitness tracking applications that integrate with the nutrition analysis system. The third-party fitness trackers 180 communicate with the application server 130 through application programming interfaces provided by fitness tracker manufacturers or health data platforms such as Apple HealthKit, Google Fit, or similar services. The third-party fitness trackers 180 provide real-time activity data including steps taken, active minutes, heart rate, heart rate variability, sleep duration, sleep quality, and energy expenditure calculated from movement sensors and heart rate monitors. The integration with third-party fitness trackers 180 enables dynamic adjustments to meal score calculations based on real-time activity levels and biometric data.

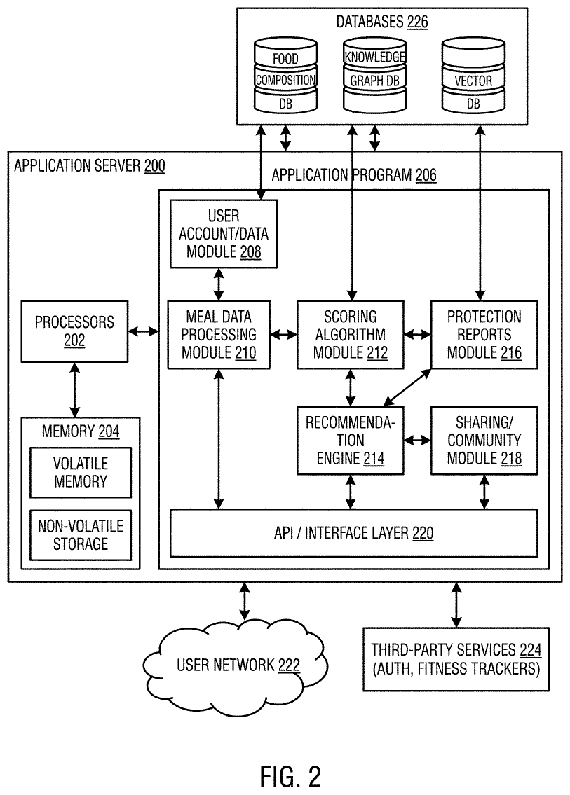

FIG. 2 illustrates a detailed block diagram of the application server architecture 200 showing the internal module organization and data flow pathways for the nutrition analysis platform, according to some embodiments. The application server architecture 200 depicts the software components and their interconnections that collectively implement the AI-driven nutrition analysis and optimal meal scoring functionality.

The application server 200 is depicted as a large rectangular boundary encompassing the various processing modules and memory components. The application server 200 represents the server-side infrastructure that receives meal data from user computing devices, processes the data through multiple analysis modules, and returns personalized meal scores and recommendations.

Processors 202 are shown at the left side of the application server 200 and comprise one or more central processing units, graphics processing units, or specialized accelerators configured to execute the computer-executable instructions of the application program. The processors 202 coordinate the execution of the various software modules and manage the flow of data between components. The processors 202 are coupled to memory 204 for accessing stored data and instructions during processing operations.

Memory 204 is depicted adjacent to the processors 202 and includes volatile memory and non-volatile storage components. The volatile memory within memory 204 provides temporary storage for data being actively processed, including meal composition data, intermediate calculation results, and user session state. The non-volatile storage within memory 204 provides persistent storage for the application program code, configuration data, and cached information that persists across system restarts.

The application program 206 is depicted within the application server 200 and encompasses the functional modules that implement the nutrition analysis capabilities. The application program 206 may be implemented using a microservices architecture where discrete functional components communicate through application programming interfaces, enabling independent scaling and deployment of individual modules based on computational demand.

A user account and data module 208 is shown within the application program 206 and manages user profile information including biometric data, health goals, dietary preferences, and activity levels. The user account and data module 208 handles user authentication, profile creation, and maintenance of personalized settings. The user account and data module 208 stores and retrieves user-specific data required for personalizing meal score calculations and generating tailored recommendations.

A meal data processing module 210 is depicted within the application program 206 and receives raw meal data input from users. The meal data processing module 210 parses and validates meal composition information including ingredients, quantities, preparation methods, and consumption timing. The meal data processing module 210 performs ingredient identification through text matching against the food composition database, barcode lookup, or image recognition processing. The meal data processing module 210 normalizes ingredient data into standardized formats suitable for downstream analysis by the scoring algorithm module.

A scoring algorithm module 212 is shown within the application program 206 and implements the multi-factor algorithm for generating personalized meal scores. The scoring algorithm module 212 evaluates ingredient quality, food processing level, nutrient density, macronutrient and micronutrient balance, glycemic index, and meal timing. The scoring algorithm module 212 applies temporal metabolic weighting to adjust nutrient values based on circadian metabolic capacity and consumption time of day. The scoring algorithm module 212 calculates dynamic nutrient synergies using interaction calculations that model non-linear relationships between nutrients within the meal. The scoring algorithm module 212 applies detractor penalties based on the presence of harmful ingredients or nutrient antagonisms. The scoring algorithm module 212 integrates the various evaluation factors to produce a comprehensive personalized meal score on a scale from zero to one hundred.

A recommendation engine 214 is depicted within the application program 206 and generates actionable recommendations for improving meal nutritional quality. The recommendation engine 214 analyzes the component sub-scores produced by the scoring algorithm module 212 to identify specific areas for improvement. The recommendation engine 214 considers user dietary preferences, available ingredients, cooking skills, time constraints, and budget when formulating recommendations. The recommendation engine 214 may utilize collaborative filtering to suggest foods that similar users have found effective for improving meal scores. The recommendation engine 214 generates specific food swap suggestions where lower-scoring ingredients may be replaced with higher-scoring alternatives serving similar culinary functions.

A Protection Reports module 216 is shown within the application program 206 and generates reports that analyze food brands, nutrition science literature, and market trends. The Protection Reports module 216 utilizes natural language processing to analyze product packaging claims and marketing materials. The Protection Reports module 216 compares stated benefits against scientific literature stored in the vector database to generate validity indices quantifying alignment between claims and evidence. The Protection Reports module 216 identifies potentially misleading marketing practices and health risks associated with specific ingredients. The Protection Reports module 216 evaluates emerging dietary trends and assesses the scientific basis for popular nutritional claims.

A sharing and community module 218 is depicted within the application program 206 and enables users to share meal scores, meal cards, and Protection Reports with designated individuals or communities. The sharing and community module 218 supports transmission to accountability partners, family members, healthcare providers, or nutrition coaches through secure messaging, email, or integration with third-party communication platforms. The sharing and community module 218 manages privacy settings that control what information is shared and with whom. The sharing and community module 218 supports real-time updates and accountability scoring to track user adherence to personalized meal plans.

An API and interface layer 220 is shown at the bottom of the application program 206 and provides the communication interface between the application server 200 and external systems. The API and interface layer 220 exposes application programming interfaces for the user computing devices to submit meal data and receive scores and recommendations. The API and interface layer 220 handles authentication, request validation, rate limiting, and response formatting. The API and interface layer 220 also provides interfaces for integration with third-party services including fitness trackers and authentication providers.

A user network 222 is depicted below the application server 200 and represents the network connection through which user computing devices communicate with the API and interface layer 220. The user network 222 corresponds to the user network 120 shown in FIG. 1 and provides the communication pathway for meal data submission and score delivery.

Third-party services 224 are shown adjacent to the user network 222 and include authentication providers and fitness tracker integrations. The third-party services 224 communicate with the API and interface layer 220 to provide authentication capabilities and real-time biometric data. The third-party services 224 correspond to the third-party authentication services 170 and third-party fitness trackers 180 shown in FIG. 1.

Databases 226 are depicted at the top of the diagram and include the food composition database, knowledge graph database, and vector database. The databases 226 are in operable communication with the application program 206 modules to provide nutritional data, interaction relationship data, and scientific literature for analysis. The food composition database within databases 226 corresponds to the food composition database 140 shown in FIG. 1. The knowledge graph database within databases 226 corresponds to the knowledge graph database 150 shown in FIG. 1. The vector database within databases 226 corresponds to the vector database 160 shown in FIG. 1.

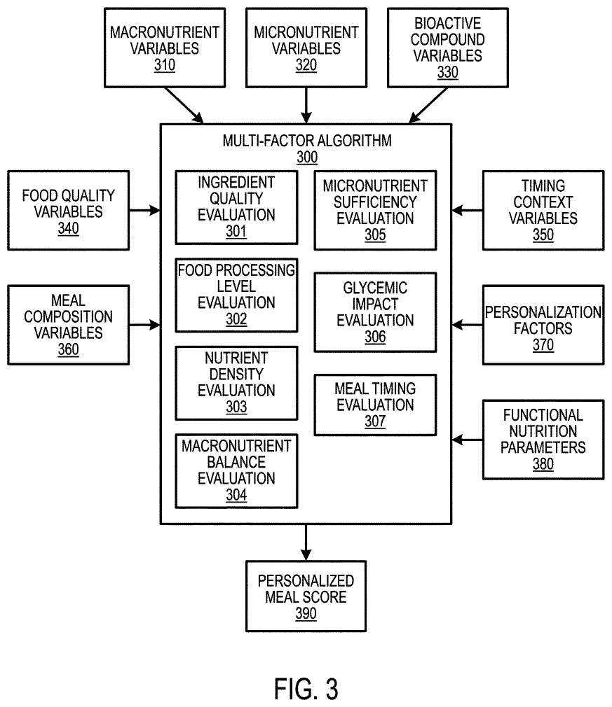

FIG. 3 illustrates a schematic diagram of the multi-factor algorithm architecture 300 depicting the variable categories and evaluation modules used to generate personalized meal scores, according to some embodiments. The multi-factor algorithm 300 integrates multiple data inputs and evaluation functions to produce a comprehensive assessment of meal nutritional quality.

Macronutrient variables 310 are depicted at the upper left portion of the diagram and represent the first category of input variables processed by the multi-factor algorithm 300. The macronutrient variables 310 include total protein, complete protein score, essential amino acid profile, branched-chain amino acid content, leucine threshold for muscle protein synthesis, total carbohydrates, fiber content, sugar content, added sugar content, glycemic index, glycemic load, total fat, saturated fat, monounsaturated fat, polyunsaturated fat, omega-3 fatty acid content, omega-6 fatty acid content, and omega-6 to omega-3 ratio. The macronutrient variables 310 provide the foundation for evaluating the energy-providing components of the meal.

Micronutrient variables 320 are shown at the upper center of the diagram and represent essential vitamins and minerals evaluated by the algorithm. The micronutrient variables 320 include vitamins A, C, D, E, K, B1, B2, B3, B5, B6, B7, B9, and B12, as well as minerals including calcium, magnesium, potassium, sodium, iron, zinc, copper, manganese, selenium, phosphorus, iodine, and chromium. The micronutrient variables 320 are compared against reference daily intakes, estimated average requirements, recommended dietary allowances, adequate intakes, and tolerable upper intake levels, with specific reference values adjusted based on user age, sex, and physiological status.

Bioactive compound variables 330 are depicted at the upper right of the diagram and represent non-nutrient food components with biological activity. The bioactive compound variables 330 include polyphenols, flavonoids, carotenoids, glucosinolates, organosulfur compounds, and prebiotic fibers. The bioactive compound variables 330 capture the health-promoting compounds found in whole foods that extend beyond traditional nutrient analysis.

Food quality variables 340 are shown at the left side of the diagram, feeding into the multi-factor algorithm 300. The food quality variables 340 assess sourcing information such as organic certification, grass-fed designation for animal products, wild-caught designation for seafood, and geographic origin. The food quality variables 340 enable the algorithm to differentiate between foods of varying quality levels within the same food category.

Timing context variables 350 are depicted at the right side of the diagram and represent temporal factors affecting nutrient utilization. The timing context variables 350 include the date and time of meal consumption, the user's chronotype, and any circadian disruptions such as shift work or jet lag. The timing context variables 350 enable the application of temporal metabolic weighting to adjust nutrient values based on circadian metabolic capacity and consumption time of day.

Meal composition variables 360 are shown at the lower left of the diagram and represent the structural characteristics of the meal. The meal composition variables 360 include the number and types of food items, portion sizes, and ingredient combinations. The meal composition variables 360 provide input for calculating dynamic nutrient synergies and cascading bioavailability effects based on food combinations within the meal.

Personalization factors 370 are depicted at the lower right of the diagram and represent user-specific characteristics that modify scoring calculations. The personalization factors 370 include biometric data, health goals, dietary preferences, activity levels, and current metabolic state. The personalization factors 370 enable the algorithm to tailor assessments to individual user needs and circumstances.

Functional nutrition parameters 380 are shown adjacent to the personalization factors 370 and represent system-level health impact assessments. The functional nutrition parameters 380 map nutrients to functional engineering vectors comprising assimilation, defense, transport, biotransformation, energy, communication, and structure functional categories. The functional nutrition parameters 380 enable calculation of synergistic pathway effects between functional systems to determine systemic health impacts of the meal composition.

Within the multi-factor algorithm 300, an ingredient quality evaluation module 301 assesses the quality attributes of food items based on sourcing, certification, and origin information from the food quality variables 340. A food processing level evaluation module 302 categorizes foods according to a classification system that distinguishes between unprocessed or minimally processed foods, processed culinary ingredients, processed foods, and ultra-processed foods. The food processing level evaluation module 302 applies scoring penalties for ultra-processed foods based on the presence of industrial food manufacturing ingredients.

A nutrient density evaluation module 303 calculates the ratio of beneficial nutrients to caloric content, accounting for vitamins, minerals, fiber, essential fatty acids, and phytonutrients per calorie. A macronutrient balance evaluation module 304 evaluates macronutrient intake against user-specific targets for protein, carbohydrates, and fats, with targets adjusted based on user health goals, activity levels, and metabolic state.

A micronutrient sufficiency evaluation module 305 assesses micronutrient intake by comparing against reference values adjusted for user demographics and physiological status. A glycemic impact evaluation module 306 evaluates the glycemic index and glycemic load of carbohydrate-containing foods, with adjustments for fiber, protein, and fat that may moderate glycemic response. A meal timing evaluation module 307 applies temporal metabolic weighting using circadian multipliers based on time-dependent metabolic capacity.

The multi-factor algorithm 300 integrates outputs from all evaluation modules to generate a personalized meal score 390. The personalized meal score 390 is a numerical value on a scale from zero to one hundred that represents the overall nutritional quality of the meal as assessed against the user's personalized health profile. The personalized meal score 390 may be decomposed into component sub-scores representing different aspects of nutritional quality for detailed feedback presentation.

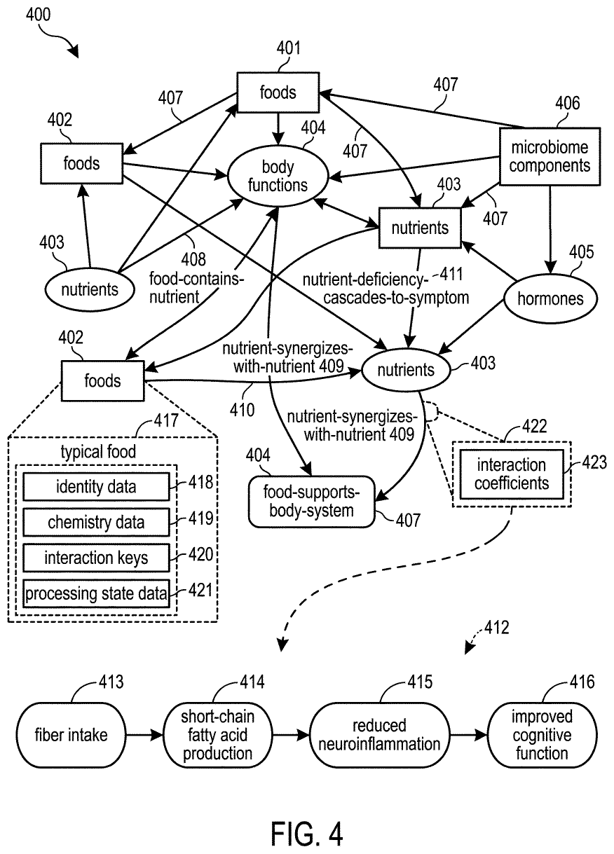

FIG. 4 illustrates a knowledge graph database schema diagram 400 showing the node types, relationship edges, and data structures used for storing and querying nutritional interaction data, according to some embodiments. The knowledge graph database schema 400 depicts the interconnected network of biological entities and their relationships that enable sophisticated nutritional analysis and recommendation generation.

At reference numeral 401, a foods node cluster is depicted at the top center of the diagram, representing the primary entry point for meal data into the knowledge graph. The foods nodes 401 represent individual food items that users may include in their meals, with each node containing comprehensive nutritional and interaction data.

Foods nodes 402 are shown at the left side of the diagram with detailed internal structure. Each foods node 402 represents a typical food item entry in the knowledge graph database. The foods nodes 402 are structured as multi-dimensional objects containing multiple data categories that enable comprehensive nutritional analysis and interaction modeling.

Nutrients nodes 403 are depicted at multiple locations throughout the diagram, representing vitamins, minerals, and other nutritional components. The nutrients nodes 403 are connected to foods nodes through food-contains-nutrient relationships indicated at reference numeral 408. The nutrients nodes 403 are also interconnected through nutrient-synergizes-with-nutrient relationships indicated at reference numeral 409, representing the synergistic and antagonistic interactions between different nutrients.

Body functions nodes 404 are shown at the upper center of the diagram, representing physiological processes and functional systems within the human body. The body functions nodes 404 are organized according to the functional engineering vectors comprising assimilation, defense, transport, biotransformation, energy, communication, and structure functional categories. The body functions nodes 404 are connected to foods nodes through food-supports-body-system relationships indicated at reference numeral 410.

Hormones nodes 405 are depicted at the right side of the diagram, representing endocrine signaling molecules that mediate metabolic responses to nutrient intake. The hormones nodes 405 are connected to nutrients nodes and body functions nodes through relationship edges representing the regulatory effects of hormones on nutrient metabolism and physiological function.

Microbiome components nodes 406 are shown at the upper right of the diagram, representing bacterial species and their metabolites that reside in the human gastrointestinal tract. The microbiome components nodes 406 enable modeling of the gut-brain axis and other microbiome-mediated health effects. The microbiome components nodes 406 are connected to foods nodes, nutrients nodes, and body functions nodes through relationship edges representing the interactions between dietary intake, gut microbiota, and physiological outcomes.

Relationship edges 407 are depicted as connecting lines throughout the diagram, representing the physiological relationships between nodes. The relationship edges 407 store associated interaction coefficients that quantify the strength and direction of relationships. The relationship edges 407 enable multi-hop queries that trace chains of causation through the knowledge graph, such as fiber intake leading to short-chain fatty acid production by gut bacteria leading to reduced neuroinflammation leading to improved cognitive function.

The food-contains-nutrient relationship type 408 is indicated between foods nodes and nutrients nodes, representing the nutritional composition of foods. The nutrient-synergizes-with-nutrient relationship type 409 is indicated between nutrients nodes, representing beneficial combinations such as vitamin C enhancement of iron absorption and piperine enhancement of curcumin bioavailability. The food-supports-body-system relationship type 410 is indicated between foods nodes and body functions nodes, representing the functional health benefits of foods.

A nutrient-deficiency-cascades-to-symptom relationship type 411 is shown connecting nutrients nodes to downstream health outcome nodes, representing the cascading effects of nutrient deficiencies on physiological function. The relationship type 411 enables the system to identify potential health consequences of dietary patterns and generate targeted recommendations for addressing nutritional gaps.

A pathway trace example 412 is depicted at the lower portion of the diagram, illustrating a multi-hop query through the knowledge graph. The pathway trace 412 demonstrates how the system traces chains of causation from dietary inputs to health outcomes. In the illustrated example, fiber intake 413 leads to short-chain fatty acid production 414 by gut bacteria, which leads to reduced neuroinflammation 415, which leads to improved cognitive function 416. This pathway trace capability enables the system to generate explanations for recommended dietary changes based on mechanistic understanding of nutrient-health relationships.

Within the typical food node structure 417, identity data 418 includes taxonomic classification and origin information for the food item. Chemistry data 419 includes nutrient density, glycemic index, bioactive compound concentrations, and physical properties affecting digestion and absorption. Interaction keys 420 define required synergies where the presence of another food or nutrient enhances bioavailability, as well as triggered antagonisms where another food or nutrient interferes with absorption. Processing state data 421 defines different bioavailability characteristics for raw versus cooked preparation states, enabling the system to account for the effects of cooking on nutrient availability.

Interaction coefficients 422 are stored on relationship edges and quantify the magnitude of nutrient interactions. The interaction coefficients 423 follow saturation kinetics models where synergy benefits increase with nutrient concentrations but approach maximum values asymptotically. For example, the vitamin C enhancement of iron absorption has a coefficient of approximately three point two, and the piperine enhancement of curcumin bioavailability has a coefficient of approximately twenty.

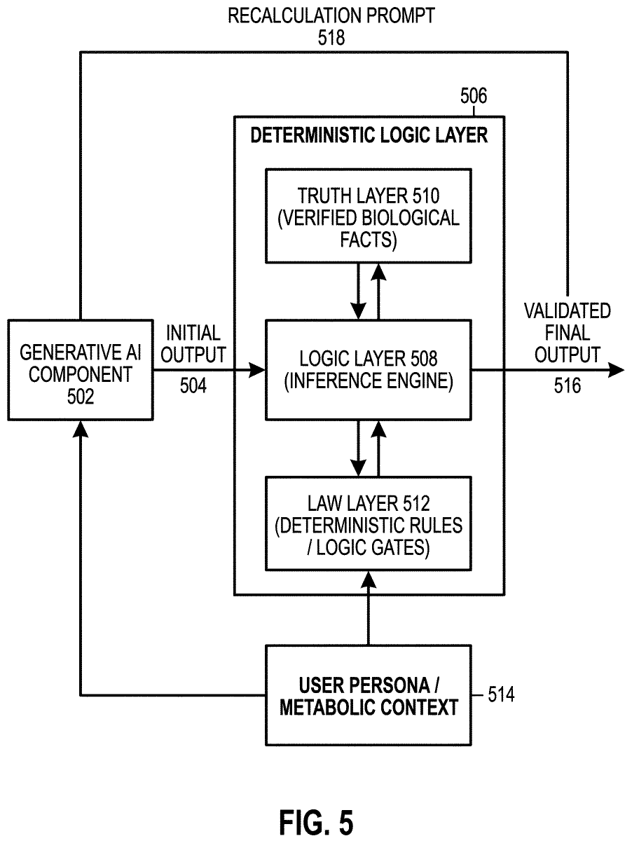

FIG. 5 illustrates a block diagram of the deterministic logic layer architecture 500 showing the triple-layer validation system for constraining AI-generated nutritional recommendations, according to some embodiments. The deterministic logic layer architecture 500 ensures that all AI-generated recommendations are validated against verified biological facts and physiological rules before being presented to users, preventing hallucination and erroneous outputs.

A generative AI component 502 is depicted at the left side of the diagram, representing the machine learning models that generate initial nutritional recommendations and meal assessments. The generative AI component 502 produces initial output 504 based on meal data, user profile information, and learned patterns from training data. The initial output 504 represents candidate recommendations that must be validated before presentation to users.

The deterministic logic layer 506 is depicted as a large rectangular boundary at the center-right of the diagram, encompassing the triple-layer validation architecture. The deterministic logic layer 506 receives initial output 504 from the generative AI component 502 and applies systematic validation to ensure recommendations comply with verified biological facts and physiological rules.

A logic layer 508, also referred to as an inference engine, is shown at the center of the deterministic logic layer 506. The logic layer 508 functions as the processor that synthesizes facts from the truth layer and rules from the law layer against user-specific metabolic context. The logic layer 508 implements the validation algorithms that cross-reference AI-generated recommendations against verified data and rules. The logic layer 508 determines whether initial outputs pass validation or require recalculation.

A truth layer 510 is depicted above the logic layer 508 within the deterministic logic layer 506. The truth layer 510 contains verified biological facts stored as read-only data that cannot be modified by the generative AI components. The truth layer 510 functions as a ground truth database containing verified biological facts derived from clinical literature, peer-reviewed research, and established scientific consensus. The truth layer 510 is designed to be versioned like software, allowing the system to incorporate new scientific consensus without disrupting the core logic. When scientific understanding evolves, the truth layer 510 may be updated to a new version that reflects current consensus.

A law layer 512 is depicted below the logic layer 508 within the deterministic logic layer 506. The law layer 512 contains deterministic rules encoded as logic gates that override any generative output that violates physiological constraints. The law layer 512 comprises a hierarchy of physiological rules including universal laws, conditional laws, and environmental laws.

Universal laws within the law layer 512 govern fundamental principles of chronobiology and thermodynamics. The universal laws include the law of energy balance stating that mass change equals energy input minus energy output, the law of essentiality stating that essential nutrient intake must meet or exceed adjusted reference daily intakes, and the law of homeostasis stating that inputs trigger regulatory responses to maintain physiological equilibrium. Conditional laws within the law layer 512 are dependent on user metabolic state and include the anabolic threshold law stating that protein intake at or above approximately thirty grams per meal triggers muscle protein synthesis, the insulin sensitivity law stating that sleep debt or high stress reduces carbohydrate tolerance, and the absorption efficiency law stating that nutrient absorption is enhanced by specific synergistic pairings. Environmental laws within the law layer 512 govern toxicity thresholds and block recommendations that would exceed tolerable upper intake levels or expose users to harmful contaminant levels.

A user persona and metabolic context module 514 is shown at the bottom of the deterministic logic layer 506. The user persona and metabolic context module 514 provides user-specific information including biometric data, health goals, dietary preferences, current metabolic state, and health conditions. The user persona and metabolic context module 514 feeds into the logic layer 508 to enable personalized application of the validation rules.

A validated final output 516 is depicted at the right side of the diagram, representing recommendations that have passed validation through the deterministic logic layer 506. The validated final output 516 represents nutritional assessments and recommendations that have been confirmed to align with verified biological facts and physiological rules appropriate for the specific user context.

A recalculation prompt 518 is shown as a feedback loop from the deterministic logic layer 506 back to the generative AI component 502. The recalculation prompt 518 is triggered when initial output 504 fails validation against the truth layer 510 or law layer 512. The recalculation prompt 518 implements a hallucination filter where invalid outputs are blocked and the generative AI component 502 is prompted to generate alternative recommendations that comply with the validation constraints. For example, if the generative AI component 502 suggests raw spinach to a user with a history of kidney stones, the law layer 512 blocks this output because raw spinach contains high oxalate content that may exacerbate kidney stone formation, and the recalculation prompt 518 directs the generative AI component 502 to suggest an alternative food.

The triple-layer architecture of the deterministic logic layer 506 prevents hallucination by ensuring that all AI-generated recommendations are validated against verified facts before being presented to users. This architecture constitutes a specific technical solution for constraining AI-generated outputs that maintains the flexibility and personalization capabilities of generative AI while ensuring safety and accuracy through deterministic validation.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrases “computing device” or “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

I/We claim:

1. A system for AI-driven nutrition analysis and optimal meal scoring, the system comprising:
at least one user computing device in operable connection with a user network;
an application server in operable communication with the user network, the application server configured to host an application program for generating personalized meal scores and providing nutritional guidance, the application program in operable communication with one or more processors coupled to a memory, wherein the one or more processors are configured to:
enable a user to create a personalized account and input personal data, including biometric data, health goals, dietary preferences, and activity levels;
receive and process meal data input by the user, the meal data comprising ingredients, preparation methods, and meal timing;
generate a meal score based on a multi-factor algorithm, wherein the algorithm evaluates at least ingredient quality, food processing level, nutrient density, macronutrient and micronutrient balance, glycemic index, and meal timing;
apply temporal metabolic weighting to adjust nutrient values based on circadian metabolic capacity and consumption time of day;
calculate dynamic nutrient synergies using interaction calculations that model non-linear relationships between nutrients within the meal;
provide real-time feedback and actionable recommendations to the user for improving the nutritional quality of the meal based on the generated meal score;
generate and display Protection Reports that analyze food brands, nutrition science literature, and market trends to provide users with insights regarding the health impact and marketing claims of selected food items; and
enable users to share meal scores, meal cards, and Protection Reports with designated individuals or communities to facilitate accountability and engagement in nutritional goals.

2. The system of claim 1, wherein the application program includes an interactive user interface that presents meals as visual meal cards, each meal card displaying nutritional highlights, meal scores, and suggested improvements for increasing the meal score.

3. The system of claim 1, wherein the application program integrates with third-party fitness trackers and wearable devices to dynamically adjust meal score calculations based on real-time activity levels and biometric data.

4. The system of claim 1, wherein the Protection Reports are generated periodically or on demand and include analysis of ingredient sourcing, food additives, and potential health risks associated with consumption of specific food items or brands.

5. The system of claim 1, wherein the multi-factor algorithm utilizes a comprehensive variable system comprising at least 120 variables across categories including macronutrients, micronutrients, bioactive compounds, food quality, timing context, meal composition, personalization factors, and functional nutrition parameters.

6. The system of claim 1, wherein the one or more processors are further configured to apply cascading bioavailability functions that calculate nutrient availability based on consumption sequence of foods within the meal.

7. The system of claim 1, wherein the one or more processors are further configured to adapt the meal score based on an adaptive personal metabolic state that incorporates current metabolic state variables including glucose levels, recent exercise activity, sleep quality, and stress indicators.

8. The system of claim 1, further comprising a knowledge graph database structured to store biological entities as nodes and physiological relationships as edges, wherein the knowledge graph enables multi-hop queries for determining nutrient interaction effects and synergistic relationships between food components.

9. A computer-implemented method for AI-driven nutrition analysis and optimal meal scoring, the method comprising:
receiving, by one or more processors, user profile data comprising biometric data, health conditions, dietary preferences, fitness goals, and activity levels;
receiving, by the one or more processors, meal data input by a user, the meal data comprising a plurality of ingredients, preparation methods, and a meal consumption time;
applying, by the one or more processors, temporal metabolic weighting functions to adjust nutritional values of the plurality of ingredients based on circadian rhythm optimization and the meal consumption time;
calculating, by the one or more processors, dynamic nutrient synergies between the plurality of ingredients using a synergy matrix that models non-linear nutrient interaction effects;
generating, by the one or more processors, a personalized meal score based on evaluation of at least ingredient quality, food processing level, nutrient density, macronutrient balance, micronutrient sufficiency, glycemic impact, and the calculated dynamic nutrient synergies;
applying, by the one or more processors, detractor penalties to the personalized meal score based on presence of identified harmful ingredients or nutrient antagonisms within the meal;
providing, by the one or more processors, real-time feedback to the user comprising the personalized meal score and actionable recommendations for improving nutritional quality of the meal; and
storing the meal data and associated personalized meal score in a database for tracking user dietary patterns over time.

10. The method of claim 9, wherein applying temporal metabolic weighting functions comprises calculating circadian multipliers for different nutrient classes based on time-dependent metabolic capacity, including increased protein value during morning hours corresponding to muscle synthesis peaks and decreased carbohydrate value during evening hours corresponding to reduced insulin sensitivity.

11. The method of claim 9, wherein calculating dynamic nutrient synergies comprises applying interaction calculations for specific nutrient pairs including vitamin C enhancement of iron absorption, piperine enhancement of curcumin bioavailability, and fat-soluble vitamin absorption enhancement in the presence of dietary fats.

12. The method of claim 9, further comprising:
generating, by the one or more processors, a Protection Report analyzing food brands and ingredients within the meal data against nutrition science literature to identify potentially misleading marketing claims or health risks associated with specific food items.

13. The method of claim 9, further comprising:
applying, by the one or more processors, cascading bioavailability functions that optimize nutrient absorption calculations based on eating sequence, wherein consumption of fiber prior to proteins and carbohydrates results in modified glycemic impact values.

14. The method of claim 9, further comprising:
adapting, by the one or more processors, the personalized meal score based on real-time user metabolic state data received from integrated wearable devices, wherein the metabolic state data comprises activity expenditure, sleep metrics, and physiological indicators.

15. The method of claim 9, further comprising:
transmitting, by the one or more processors, the personalized meal score and meal data to designated accountability partners, family members, or healthcare providers selected by the user for progress monitoring and collaborative nutritional planning.

16. A non-transitory computer-readable storage medium storing computer-executable instructions that, when executed by one or more processors, cause the one or more processors to perform operations for AI-driven nutrition analysis and optimal meal scoring, the operations comprising:
receiving user profile data comprising biometric data, health goals, dietary preferences, and health conditions;
receiving meal composition data comprising a plurality of food items, ingredient quantities, preparation methods, and a meal consumption time;
retrieving, from a knowledge graph database, nutritional data and interaction relationship data for each of the plurality of food items, wherein the knowledge graph database comprises food nodes with associated nutrient attributes and edge relationships defining nutrient synergies and antagonisms;
applying a deterministic logic layer to constrain nutritional analysis outputs, wherein the deterministic logic layer comprises a hierarchy of physiological rules including universal laws governing chronobiology and thermodynamics, conditional laws dependent on user metabolic state, and environmental laws governing toxicity thresholds;
calculating a personalized meal score by integrating temporal metabolic weighting based on the meal consumption time, dynamic nutrient synergy calculations derived from the knowledge graph database, adaptive personal metabolic state adjustments, and cascading bioavailability functions based on food consumption sequence;
generating actionable recommendations for improving the personalized meal score based on identified nutrient deficiencies, suboptimal ingredient combinations, or meal timing adjustments; and
presenting the personalized meal score and actionable recommendations to a user through an interactive user interface comprising visual meal cards that display nutritional highlights and suggested food swaps.

17. The non-transitory computer-readable storage medium of claim 16, wherein the operations further comprise:
generating a Protection Report that analyzes nutrition science literature, brand claims, and emerging dietary trends to provide the user with transparent and unbiased insights into food choices, including warnings about potentially misleading marketing practices or health risks associated with specific ingredients.

18. The non-transitory computer-readable storage medium of claim 16, wherein the knowledge graph database comprises food nodes structured as multi-dimensional objects containing identity data, chemistry data including nutrient density and glycemic index, interaction keys defining required synergies and triggered antagonisms, and processing state data defining different bioavailability characteristics for raw versus cooked preparation states.

19. The non-transitory computer-readable storage medium of claim 16, wherein the deterministic logic layer comprises a triple-layer architecture including a truth layer containing verified biological facts as read-only data, a law layer containing deterministic rules encoded as logic gates for validating outputs, and a logic layer comprising an inference engine that synthesizes the truth layer and law layer against user-specific metabolic context.

20. The non-transitory computer-readable storage medium of claim 16, wherein the operations further comprise:
mapping standard anatomical systems to functional engineering vectors comprising assimilation, defense, transport, biotransformation, energy, communication, and structure functional categories; and
calculating synergistic pathway effects between functional systems to determine systemic health impacts of the meal composition, including gut-brain axis interactions affecting cognitive function and detox-defense axis interactions affecting inflammatory response.
